(19) United States
(12) Patent Application Publication
(10) Pub. No.: US 2007/0134299 A1
(43) Pub. Date: Jun. 14, 2007

(54) Method and formula for prevention of cold and flu and suppression of related symptoms

(76) Inventor: Brian C. Giles, Montecito, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/296,879

(22) Filed: Dec. 8, 2005

(51) Int. Cl. A61K 36/8962; A61K 33/24; A61K 36/537; A61K 36/53; A61K 36/534; A61K 33/00; A61K 36/81

(52) U.S. Cl. 424/440; 424/617; 424/722; 424/464; 424/745; 424/746; 424/747; 424/725; 424/754; 424/760; 424/451

(57) ABSTRACT

The present invention generally relates to methods, compositions and systems for preventing the common cold and flu, for reducing the duration of common colds and flu, for reducing the severity of common cold and flu symptoms and for reducing/eliminating the transmission of the common cold and flu. Specifically, it relates to the treatment a of subject having a cold with a composition comprising cesium and/or rubidium ions. In a composition aspect, the present invention provides a composition for treating a common cold and flu.

### FIELD OF THE INVENTION

The present invention generally relates to a methods, compositions and systems for preventing and reducing the duration of common colds and flu and for reducing the severity of common cold and flu symptoms.

### BACKGROUND OF THE INVENTION

The mouth, like the rest of the outside surfaces of the body, contains a very large population of bacteria (micro-organisms) which live there all the time. Viruses that cause the common cold, for example, may be spread through the air as aerosols in the form of a sneeze or cough. Additionally, the viruses are capable of surviving outside of a host for extended periods of time. Transmission through common contact (e.g., shaking hands or handling contaminated objects) is easily transmitted from host to host. The gravity of this fact is increased in that the effect of a virus or viruses may be variable; what causes a cold in one subject may cause a fatal, upper respiratory tract infection in another.

A considerable body of information has been acquired on the etiology and epidemiology of the common cold and variety of flu viruses. It is known, for instance, that the common cold is a group or family of diseases caused by a number of viruses. Such viruses, or the family, include, but are not limited to adenoviruses, parainfluenza viruses, influenza viruses, rhinoviruses, respiratory syncytial viruses, coronaviruses, echoviruses, coxsackieviruses, and enteroviruses. The molecular biology of rhinovirus, which causes at least 50% of all upper respiratory tract infections, is understood in great detail. Several environmental factors that dictate the survival of these viruses are, furthermore, known.

Despite the accumulated knowledge on these viruses, progress on the effective treatments for the common cold and flu has been slow and still remain ineffective.

There are several over-the-counter compositions available as common cold and flu remedies. These products typically include antihistamines, decongestants, pain relievers (e.g., aspirin, acetaminophen, and ibuprofen), cough suppressants, expectorants or analgesics. They generally do not reduce the duration of a cold or flu, and often they are accompanied by undesirable side effects. In short, these compositions mask symptoms rather than treat the underlying cause.

Zinc ions have been reported to inhibit the replication of rhinoviruses. See Korant, B. D. et al., Nature 248:588-590 (1974). Subsequent to the report, soluble and ionizable zinc compounds have been used to treat colds through their application to oral and oral pharyngeal mucosa. Some studies have indicated a shorter cold duration when this treatment has been used, however the data is still being debated.

There is accordingly a need for improved methods and compositions for prevention (prophylaxis) and or reducing the duration of common colds and flu and for reducing the severity of common cold and flu symptoms. That is an object of the present invention.

### SUMMARY OF THE INVENTION

The present invention generally relates to methods, compositions and systems for preventing (prophylaxis) and/or reducing the duration of common colds and flu and for reducing the severity of common cold and flu symptoms. Specifically, it relates to the treatment a of subject having a cold or flu with a composition including cesium and/or rubidium ions.

In a composition aspect, the present invention provides a composition for treating a common cold. The composition includes 50 ppm to 500,000 ppm of a cesium or rubidium ion source.

In another composition aspect, the present invention provides a composition for treating the flu. The composition includes 50 ppm to 500,000 ppm of a cesium or rubidium ion source.

In another composition aspect, the present invention provides a composition for preventing a cold. The composition includes 50 ppm to 500,000 ppm of a cesium or rubidium ion source.

In another composition aspect, the present invention provides a composition for preventing the flu. The composition includes 50 ppm to 500,000 ppm of a cesium or rubidium ion source.

In some cases, the composition further includes a structuring agent selected from gum tragacanth, xanthan gum, gum karaya and gum arabic, seaweed derivatives, smectite clays such as diatomaceous earths, bentonite or hectorite, calcium apatite, carboxyvinyl polymers and water-soluble cellulose derivatives such as hydroxyethyl cellulose and sodium carboxymethyl cellulose present in an amount of from 0 to 5 percent by weight of the composition.

In other cases, the composition further includes a flavoring agent selected from wintergreen oil, oregano oil, bay leaf oil, cherry oil, peppermint oil, spearmint oil, clove oil, sage oil, sassafras oil, lemon oil, lime oil, orange oil, anise oil, benzaldehyde, bitter almond oil, camphor, cedar leaf oil, marjoram oil, citronella oil, lavender oil, mustard oil, pine oil, pine needle oil, rosemary oil, thyme oil, cinnamon leaf oil, and mixtures thereof present in an amount from 0.05 to 5 percent by weight of the composition.

In other cases, the composition further includes an ingredient selected from Allium cepa (red onion), Salvia officinalis (Sage), Sambucus nigra (elderberry), Capsicum (cayenne fruit), Mentha pipenta (peppermint), Thymus serpyllum (thyme), Aconitum napellus (monks hood), Allium sativum (garlic), Anas barbariae (Oscillococcinum), Euphrasia officinalis (eyebright), Gelsemium sempervirens (yellow jasmine), Phytolacca decandra (poke), and Pulsatilla nigricans (wind flower) present at a concentration of 1× to 60 C.

In other cases, the composition further includes a drug or drugs selected from antihistamines, decongestants, pain relievers, cough suppressants, expectorants, and analgesics.

In other cases, the composition further includes either elemental zinc or a zinc salt selected from zincum gluconicum, zinc gluconate, and zinc sulfate present in a concentration ranging from 0.1 percent to 3.0 percent weight/volume of the composition.

In a formulation aspect, the present invention provides a composition of the present invention formulated as a capsule, tablet, lozenge, troche, hard candy, powder, spray, mouth rinse, gel, paste, elixir, gum, syrup or suspension.

In a method aspect, the present invention provides a method of treating a subject who has (i.e., feels increasing symptoms) or anticipates a cold or flu. The method involves treating the subject with a composition or formulation of the present invention.

### DETAILED DESCRIPTION OF THE INVENTION

The term “acid” refers to a substance consisting of molecules of ions which donate protons (H+), and a base is a substance which accepts protons.

The terms “acidity” and “alkalinity” refer to values measured by pH, which is defined as the logarithm of the Hydrogen ion activity: pH=−log (H). The parameter pHe is the pH on the exterior and pHi is the pH on the interior of the cell.

The term “acid-forming reaction” refers to any chemical reaction that produces a decreased ability to energize the biological system and leaves an acid residue, such as a hydrogen ion (H+). The result is localized acidosis with induced hypoxia, which is a major cause of a wide variety of disease states (cold and flu environment).

The term “reducing activity” or “to reduce the acidity of” as used herein in relation to the tissue of a mammal means to elevate and/or maintain the optimum or near optimum pH, pHe and pHi ranges of a surface or a portion of tissues such that various pH's are above the action potentials responsible for nerve conduction (i.e., 6.80 or higher).

The term “common cold,” or simply “cold,” refers to an upper respiratory illness caused by a wide variety of viral infections. Colds include a number of symptoms, including nasal drainage, nasal congestion, headache, watery eyes, fever, aches, pains, dryness, myalgia, sneezing, sore throat, scratchy throat, cough, hoarseness, and bronchial-sinusitis symptoms.

The term “flu” refers to at least three known influenza-type viruses that affect human beings: Influenza A, B and C. Influenza A viruses have been isolated from many animal species in addition to humans, while the influenza B and C viruses infect mainly humans. The influenza viruses are enveloped viruses containing negative single-stranded RNA's which are segmented and encapsidated. The influenza virus envelope is characterized by the presence of two surface glycoproteins: hemaglutinin and neuraminidase. The influenza A and B virions are pleomorphic and are usually 80-120 mn in diameter. The influenza C virion has many distinctive properties and is thus distinguished from the closely related A and B virions. Infection with influenza A or B often can cause a highly contagious, air-born respiratory illness.

The term “effective amount” refers to an amount or quantity of an active substance that is sufficient to elicit the required or desired therapeutic gain or response.

The term “mammal” has its normal dictionary meaning. Specific examples of mammals include humans, dogs, cats, horses, and cows.

The term “subject who is suffering from a cold or flu” is equivalent to a “subject who is suffering from the symptoms of a cold or flu.” Both terms refer to a subject who is experiencing the above-listed, common cold or flu symptoms or a combination.

The term “subject in need of treatment” includes subjects who have contracted a virus that may cause a common cold, subjects who exhibit symptoms of a common cold or flu, subjects who are suffering from a cold or flu or from the symptoms of a cold or flu, and those subjects who wish to take preventative measures to avoid infection from a wide variety of common cold and flu inducing viruses.

The terms “treatment” and “prophylaxis” are used in a broad sense and extend from symptomatic cold and flu relief to the elimination of infection to general preventative therapy for cold and flu.

Human health can be influenced by many factors. One of the most fundamental factors is the correct function of homeostatic regulation of ionic concentrations, pH, ORP, and membrane electrical potentials. Biochemical activities of living cells are regulated by pH and ion concentrations including many key processes, such as energy metabolism and nerve and brain function, depend on membrane electrical potential. It is also known that ion movements, correct pH and membrane electrical potential are closely linked through the action of ion specific gates, electrogenic carriers and other ion carriers in cell and organelle membranes.

Cold and flu viruses invasion and propagation are caused (requires) or promoted by a reduction of the optimum pH, pHe and pHi ranges and progress though a complex series of pathological and electro-physiological and electro-chemical changes from initial onset to full manifestation. Unfortunately, current drug compositions on the market do not currently address or understand the underlying causes and their mechanisms on an electro-physiological level of action or the complexity of these viral disease environments and processes. This makes them extremely difficult to control and eliminate without knowing their electro-chemical and electro-physical mechanisms.

There are many drugs and therapies aimed at correcting specific disease symptoms that result from the biological system loss of the critical pH, pHe and pHi, ionic, and electro-physiological conditions. Preventative protection of the biological system from failure of homeostasis has generally been relegated largely to the realm of nutritional and lifestyle choices. The knowledge and understanding of ionic physiology enables methods and compositions that will make possible the protection, maintenance and restoration of ionic homeostasis, such as reducing or eliminating a viral invasion and the resulting damage of the cells and tissues.

The aberrant regulation of cellular homeostasis is a significant factor in the pathogenesis of viral disease onset. Elevating the pH to an optimum or near optimum range, (cellular pHe and cellular pHi) normalizes and stabilizes the ionic homeostasis. As an example, the central disorders of acute maladaptive reactions are acidity and hypoxia. The biochemistry of viral disease invasion such as cold and flues reveals the same disorders as acute maladaptive reactions (sub-optimal pH), which produce an acidic biological environment.

Cesium and/or Rubidium ions provide an “electron bath.” The free radicals are bathed in electrons, and they are then stabilized and no longer able to propagate and induce cell tissue damage. The optimally functioning electro- physiological environment has a narrow pHe ranging from 6.80 to 7.55, preferably between 7.31 to 7.50. The current invention method and composition elevates the pH to optimum or near optimum ranges eliminates the H+ ions migration, produces ionic changes in the pH, pHe, pHi, and changes the ionic chemistry of the cells and tissues providing electro-physiological resistance to viral disease invasion and progression.

The biological (electro-physiological) environment of cold and flu virus has a very narrow and specific viability zone, limited to a narrow pH and ORP range. Note that the cells and tissues optimally function in a pH and ORP outside the cold and flu viruses' viability zone. The composition increases negative hydrogen ions in the body fluids, the electrons tend to move those fluids toward an ORP and pH in a range consistent with optimum physiologic aerobic metabolic function.

The composition and method enhance the ability of the patient's healthy viable cells and normal tissue to tolerate and resist a decreased pHe and to resist the transport of H+ (ions) across its membrane and provides an electro-physical barrier from cold/flu virus invasion and proliferation.

An advantage of the composition aspect of the present invention can be cost effectively administered as a stand alone therapy or as an effective adjunct in conjunction with traditional remedies.

Another advantage of the invention is to provide a therapeutic composition over a sufficient period of time to suppress or eliminate infectious microorganisms (e.g., bacteria, viruses, etc.) as related to cold and flu suppression, elimination and prophylaxis.

An object of the invention is to provide a prophylactic, composition to restore and elevate the pH, pHe and pHi for a brief period of time or longer.

A further object of the invention is to administer a composition to obtain an optimum or near optimum saliva pH, pHe and pHi values/levels for cold virus resistance and prophylaxis. This is accomplished by shifting the pHe to a value/levels ranging from 6.80 to 7.55, preferably to a value/levels ranging from 7.30 to 7.50.

A further object of the invention is to provide a effective treatment for the common cold and or flu. Specifically, it is an object of the invention to provide remedies that reduce the duration of a cold and or flu, that reduce the severity or duration of one or more symptoms of a cold or flu, and/or that prevent a subject from contacting a common cold or flu and the spreading (transmission) of the cold and or flu virus.

A further object of the invention is to overcome the limitations, problems and disadvantages associated with traditional approaches for treating the common cold and flues by manipulating the pH and ionic environment of a subject.

Compositions

The compositions of the present invention include a cesium or rubidium ion source preferably present in an amount sufficient to provide from 50 ppm to 500,000 ppm, preferably 500 ppm to 100,000 ppm, cesium and/or rubidium ions.

The salts included in the composition of the present invention may be employed in the composition of the present invention may be formed using a wide variety of acids, including, but not limited to: asporotate, orotate, aspartate, malic, succinic, etc. chloride carbonate, malic and citric acids are preferred.

Any combination of salts containing ions of cesium and/or rubidium which dissociate and ionize may be employed in the composition of the present invention, including, but not limited to: Arginate, Ascorbate, Aspartate, Acetic, Caprylate, Chloride, Carbonate, Cysteinate, Citrate, Fumarate, Humic, Fulvate, Fulvic, Methionine, Glutamate, Gluconate, Glycinate, Lysinate, Succinate, Lactic, Lactate, Malate, Tartrate, Sulfate, Phosphate, Nitrate, Fluoride, Bromide, Iodide, Orotate, Asporotate, Bisulfonate, Lysinate, Succate, Carnate, Trisulfate, Lactobionate, Benzenesulfonate, Laurate, Benzoate, Bicarbonate, Benzoic, Caseinate, Bisufate, Mandelate, Bitartrate, Mesylate, Borate, Methylbromide, Methylnitrate, Calcium Edetate, Methylsulfate, Camsylate, Mucate, Napsylate, Oleate, Edetate, Oxalate, Edisylate, Pamoate (Embonate), Esolate, Palmitate, Esylate, Pantothenate, Phosphate/Diphosphate, Gluceptate, Polygalacturonate, Salicylate, Stearate, Glycollylarsanilate, Hexylresorcinate, Subacetate, Hydrabamine, Hydrobromide, Tannate, Hydrochloric, Hydrochloride, Hydroxynaphthoate, Teoclate, Tartaric Tosylate, Isothionate, Triethiodide, Panoate, Valerate, Acetate, Maleate, Malonate, and mixtures thereof.

Additionally, other cesium and rubidium salts might be used in a wide variety of compositions, such as, but not limited to, various organic or metallic salts, if they meet the following requirements: (1) they must be pharmaceutically acceptable and have an acceptably low level of toxicity; (2) they must have sufficient high levels of cationic (alkaline) dissociation to allow the remaining negatively charged ions to effectively reduce the acidity of the tissues and tissue surfaces.

Potentiation of cesium and/or rubidium ionic action can be accomplished by inclusion of ingredients that enhance ionic pH physiology. Examples are electrolytes (saline compounds) such as potassium, sodium, and magnesium. Potassium, magnesium and other electrolytes (e.g., sodium, calcium, chloride, bicarbonate, phosphate and sulfates) are added to the composition in proportion to the potassium.

Other, optional ingredients that may be included to potentiate the activity of cesium/rubidium ionic action include manganese, zinc, boron, vitamin B2 (riboflavin), B12 (cyanocbalamin), and B6 (pyridoxine).

A solvent (e.g., water), especially in the case of a rinse or spray, is a typical, but optional ingredient of the present composition. Where the solvent is water, a preferred method of manufacturing is to use water processed by means such as E.C.A. (electro-chemical activation). The method produces aqueous solutions having certain characteristics. For example, an aqueous solution (water) for an oral treatment may be processed having a surface tension ranging from 30 to 70 dynes per cm2 producing an ORP (oxidative reduction potential) ranging from +5 m.v. to −400 m.v., preferably ranging from −10 m.v. to −250 m.v. After the ionic dissociation, the pH for such compositions ranges from 6.50 to 8.50, preferably from 6.90 to 8.40.

The compositions may contain the following ingredients, if they do not interfere with the dissociation or mobility of cesium or rubidium ions: humectants, gelling agents, desensitizing agents, flavorings, colorings, sweeteners, preservatives and structuring agents, and surfactants. Suitable surfactants are water-soluble organic compounds, and may be nonionic, cationic or amphoteric species throughout a suitable pH range.

Structuring agents may be used in, for example, dentifrices and gums to provide desirable textural properties and “mouth feel”. Suitable agents include natural gum binders such as gum tragacanth, xanthan gum, gum karaya and gum arabic, seaweed derivatives, smectite clays such as diatomaceous earths, bentonite or hectorite, calcium apatite, carboxyvinyl polymers and water-soluble cellulose derivatives such as hydroxyethyl cellulose and sodium carboxymethyl cellulose. Improved texture may also be achieved, for example, by including colloidal magnesium and or aluminum silicate. Suitably, the structuring agent is included in an amount of from 0-5%, preferably 0-3% by weight of the oral composition.

Flavoring agents may be included in the composition if desired, as long as they do not substantially interfere with the ionic mobility or dissociation of cesium and/or rubidium. Such agents—e.g. oils of peppermint, wintergreen, sassafras and clove—are typically included to increase the palatability of a composition. Flavoring agents may be lipophilic or hydrophilic. Lipophilic flavorants include, without limitation, wintergreen oil, oregano oil, bay leaf oil, peppermint oil, spearmint oil, clove oil, sage oil, sassafras oil, cherry oil, lemon oil, orange oil, anise oil, benzaldehyde, bitter almond oil, camphor, cedar leaf oil, marjoram oil, citronella oil, lavender oil, mustard oil, pine oil, pine needle oil, rosemary oil, thyme oil, cinnamon leaf oil, and mixtures thereof. Where used, lipophilic flavorants are typically included in the oral composition at a level from 0.01%-10% by weight, preferably from 0.05%-5.0% by weight, and more preferably from 0.1%-3.0% by weight.

Sweetening agents such as the following may also be included: D-tryptophan, mannitol, dextrose, levulose, acesulfam, dihydrochalcones and sodium cyclamate. Such flavoring or sweetening agents are typically included in the oral composition in an amount from 0-5% by weight, preferably 0-2% by weight. Furthermore, coloring agents (e.g., colorants or pigments) may be added to improve the visual appearance of the composition. Suitable colorants include, without limitation, dyes such as FD & C blue No.1, D & C yellow No.10 and D & C yellow No.3. A suitable and commonly used pigment is titanium dioxide, which provides a white color.

Homeopathic and/or herbal ingredients may be included in the composition. Examples of such ingredients include, without limitation, the following: Allium cepa (red onion); Sambucus nigra (elderberry); Capsicum (cayenne fruit); Mentha piperita (peppermint); Thymus serpyllum (thyme); Olea europaea (olive whole leaf); Lomatium dissectum (lomatium root); Rubis idaeus (red raspberry leaf); Commiphora myrrha (myrrh); Salvia officinalis (sage); Melissa officinalis (lemon oil); Tabebuia avellanedae (Pau d'arco); Glycyrrhiza glabra (licorice); Althea officinalis (marshmallow root tea); Pumonaeia officinalis (lungwort); Trigonella foenum-graecum (fenugreek); Aconitum napellus (monks hood); Allium sativum (garlic); Anas barbariae (Oscillococcinum); Euphrasia officinalis (eyebright); Gelsemium sempervirens (yellow jasmine); Phytolacca decandra (poke); and Pulsatilla nigricans (wind flower); and mixtures thereof.

Where included, Allium cepa (red onion), Salvia officinalis (Sage), Sambucus nigra (elderberry), Capsicum (cayenne fruit), Mentha pipenta (peppermint), Thymus serpyllum (thyme), Aconitum napellus (monks hood), Allium sativum (garlic), Anas barbariae (Oscillococcinum), Euphrasia officinalis (eyebright), Gelsemium sempervirens (yellow jasmine), Phytolacca decandra (poke), and Pulsatilla nigricans (wind flower) are typically present at homeopathic concentrations, preferably at a concentration of 1× to 60 C. Preparation of homeopathic ingredients is generally accomplished through successive dilutions and potentiations, and it is well within the ability of one skilled in the art of homeopathy.

When Olea europaea (olive whol leaf), Lomatium dissectum (lomatium root), Tubis idaeus (red raspberry leaf), Commiphora myrrha (myrrh), Trigonella foenumgraecum (fenugreek), Pulmonan officinalis (lungwort), Althea officinalis (marshmallow root tea), Glycyrrhiza glabra (licorice), Ulmus rubra (slippery elm bark), Tabebuia avellanedae (Pau d'arco), Thymus vulgaris (thyme), Melissa officinalis (lemon oil), Allium sativum (garlic), Capsicum annuum (cayenne fruit) are included in the compositions of the present invention, they may be present at a concentration from 0.01 to 10 percent weight/volume. Preferably, the ingredients are included at a concentration of 0.1 percent to 5 percent weight/volume of the composition, and more preferably 0.5 percent to 1 percent.

Drugs that are commonly used to treat cold symptoms may also be included in the compositions of the present invention. Classes of such drugs include, without limitation, antihistamines, decongestants, analgesics (e.g., acetaminophen), cough suppressants, expectorants, and analgesics.

Either elemental zinc or zinc salts may also be included in the compositions of the present invention. Nonlimiting examples of zinc salts include zincum gluconicum, zinc aspartate, zinc citrate, zinc gluconate, and zinc sulfate. When present, the concentration of zinc salts in the compositions typically ranges from 0.1 percent to 3.0 percent weight/volume.

For the treatment of flu, the composition of the present invention may further include compounds known to have a beneficial effect with respect to the treatment or prevention of flu. Such compounds include, without limitation, the following: amantadine; rimantadine; pyridazine derivatives reported in U.S. Pat. No. 5,935,957, which is hereby incorporated-by-reference for all purposes; 3-(3,4-dihydroxy-5-nitrobenzylidene)-2,4-pentanedione, N-methyl-D-glucamine dithiocarbamate (MGD), nitric oxide radical synthase (NOS) inhibitors, competitive inhibitor for NOS substrate, L-arginine analogues, aminoguanidine, 7-nitroindazole, S-ethylisothiourea, S-methylisothiourea, S-methylthiocitrulline, S-ethylthiocitrulline, N-ethylimino-L-omithine, flavoprotein binding inhibitors diphenyleneiodonium, tetrahydrobiopterin biosynthesis inhibitors 2,4-diamino-6-hydroxypyrimidine, inhibitors of NOS induction/generation corticosteroids such as dexamethasone, transforming growth factor(TGF)-β-1, 2 or 3, interleukin-4, interleukin-10, as discussed in U.S. Pat. No. 6,043,268, which is hereby incorporated-by-reference for all purposes; the tripeptide analog glutathione (GSH), its oxidized dimer glutathione disulfide (GSSG), ascorbate-2-phosphate, N-acetyl-L-cysteine, or combinations thereof, as described in U.S. Pat. No. 6,187,281, which is hereby incorporated-by-reference for all purposes; and compounds disclosed in U.S. Pat. No. 6,225,341, which is hereby incorporated-by-reference for all purposes.

Other optional components for use in the present compositions include: antioxidants; vitamins (e.g., vitamin C and E); pH adjusting agents; anticaries agents; plant extracts; and, mixtures thereof.

Where the composition of the present invention is used as a mouth rinse, it is preferred that the ingredients of the aqueous solution are selected such that the composition may be ingestible, even by children. For example, the aqueous solution should be free of alcohol or other ingredients that warrant poison control labeling or hazard labeling indicating that the composition is to be kept away from children. In one mouth rinse embodiment, the ingredients of the rinse composition do not include alcohol cetylpyridinium chloride or witch hazel (12-15% ethanol).

The following compositions are examples of compositions of the present invention:

Composition 1, which comprises the following: from 50 ppm to 500,000 ppm cesium ion source; and, water having a surface tension ranging from 30 to 70 dynes per cm².

Composition 2, which comprises the following: from 50 ppm to 500,000 ppm rubidium ion source; and, water having a surface tension ranging from 30 to 70 dynes per cm².

Composition 3, which comprises the following: from 50 ppm to 500,000 ppm cesium ion source; and, a structuring agent selected from gum tragacanth, xanthan gum, gum karaya and gum arabic, seaweed derivatives, smectite clays such as diatomaceous earths, bentonite or hectorite, calcium apatite, carboxyvinyl polymers and water-soluble cellulose derivatives such as hydroxyethyl cellulose and sodium carboxymethyl cellulose present in an amount of from 0 to 5 percent by weight of the composition.

Composition 4, which comprises the following: from 50 ppm to 500,000 ppm rubidium ion source; and, a structuring agent selected from gum tragacanth, xanthan gum, gum karaya and gum arabic, seaweed derivatives, smectite clays such as diatomaceous earths, bentonite or hectorite, calcium apatite, carboxyvinyl polymers and water-soluble cellulose derivatives such as hydroxyethyl cellulose and sodium carboxymethyl cellulose present in an amount of from 0 to 5 percent by weight of the composition.

Composition 5, which comprises the following: from 50 ppm to 500,000 ppm cesium ion source; and, a flavoring agent selected from wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavender oil, mustard oil, pine oil, pine needle oil, cherry oil, rosemary oil, thyme oil, cinnamon leaf oil, and mixtures thereof present in an amount from 0.05 to 5 percent by weight of the composition.

Composition 6, which comprises the following: from 50 ppm to 500,000 ppm rubidium ion source; and, a flavoring agent selected from wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavender oil, mustard oil, pine oil, pine needle oil, cherry oil, rosemary oil, thyme oil, cinnamon leaf oil, and mixtures thereof present in an amount from 0.05 to 5 percent by weight of the composition.

Composition 7, which comprises the following: from 50 ppm to 500,000 ppm cesium ion source; and, an herbal ingredient selected from Allium cepa (red onion), Salvia officinalis (Sage), Sambucus nigra (elderberry), Capsicum (cayenne fruit), Mentha pipenta (peppermint), Thymus serpyllum (thyme), Aconitum napellus (monks hood), Allium sativum (garlic), Anas barbariae (Oscillococcinum), Euphrasia officinalis (eyebright), Gelsemium sempervirens (yellow jasmine), Phytolacca decandra (poke), and Pulsatilla nigricans (wind flower) present at a concentration of 1× to 60 C.

Composition 8, which comprises the following: from 50 ppm to 500,000 ppm rubidium ion source; and, an herbal ingredient selected from Allium cepa (red onion), Salvia officinalis (Sage), Sambucus nigra (elderberry), Capsicum (cayenne fruit), Mentha pipenta (peppermint), Thymus serpyllum (thyme), Aconitum napellus (monks hood), Allium sativum (garlic), Anas barbariae (Oscillococcinum), Euphrasia officinalis (eyebright), Gelsemium sempervirens (yellow jasmine), Phytolacca decandra (poke), and Pulsatilla nigricans (wind flower) present at a concentration of 1× to 60 C.

Composition 9, which comprises the following: from 50 ppm to 500,000 ppm cesium ion source; and, an herbal ingredient selected from Olea europaea (olive whole leaf), Lomatium dissectum (lomatium root), Tubis idaeus (red raspberry leaf), Commiphora myrrha (myrrh), Trigonella foenumgraecum (fenugreek); Pulmonana officinalis (lungwort), Althea officinalis (marshmallow root tea), Glycyrrhiza glabra (licorice), Ulmus rubra (slippery elm bark), Tabebuia avellanedae (Pau d'arco), Thymus vulgaris (thyme), Melissa officinalis (lemon oil); Allium sativum (garlic), Capsicum annuum (cayenne fruit) present at a concentration of 0.1 percent to 5 percent weight/volume of the composition.

Composition 10, which comprises the following: from 50 ppm to 500,000 ppm rubidium ion source; and, an herbal ingredient selected from Olea europaea (olive whole leaf), Lomatium dissectum (lomatium root), Tubis idaeus (red raspberry leaf), Commiphora myrrha (myrrh), Trigonella foenumgraecum (fenugreek); Pulmonana officinalis (lungwort), Althea officinalis (marshmallow root tea), Glycyrrhiza glabra (licorice), Ulmus rubra (slippery elm bark), Tabebuia avellanedae (Pau d'arco), Thymus vulgaris (thyme), Melissa officinalis (lemon oil); Allium sativum (garlic), Capsicum annuum (cayenne fruit) present at a concentration of 0.1 percent to 5 percent weight/volume of the composition.

Composition 11, which comprises the following: from 50 ppm to 500,000 ppm cesium ion source; and, a non-homeopathic drug selected from antihistamines, decongestants, analgesics (e.g., acetaminophen), cough suppressants, expectorants, and analgesics.

Composition 12, which comprises the following: from 50 ppm to 500,000 ppm rubidium ion source; and, a drug selected from antihistamines, decongestants, analgesics (e.g., acetaminophen), cough suppressants, expectorants, and analgesics.

Composition 13, which comprises the following: from 50 ppm to 500,000 ppm cesium ion source; and, zinc or a zinc salt selected from elemental zinc, zincum gluconicum, zinc gluconate, zinc aspartate, zinc citrate, and zinc sulfate present in a concentration ranging from 0.1 percent to 3.0 percent weight/volume of the composition.

Composition 14, which comprises the following: from 50 ppm to 500,000 ppm rubidium ion source; and, zinc or a zinc salt selected from elemental zinc, zincum gluconicum, zinc aspartate, zinc citrate, zinc gluconate, and zinc sulfate present in a concentration ranging from 0.1 percent to 3.0 percent weight/volume of the composition.

Composition 15, which comprises the following: from 50 ppm to 500,000 ppm cesium ion source; and, one or more electrolytes selected from potassium, sodium, and magnesium.

Composition 16, which comprises the following: from 50 ppm to 500,000 ppm rubidium ion source; and, one or more electrolytes selected from potassium, sodium, and magnesium.

The compositions of the present invention may be formulated in any suitable dosage form including, but not limited to, capsules, tablets, lozenges, troches, hard candies, powders gels, pastes, elixirs, syrups, rinses, and sprays suspensions of solutions.

Typically, the compositions are delivered in a pharmaceutically acceptable carrier. Nonlimiting examples of carriers include: carbohydrates such as fructose, sucrose, sugar, dextrose, starch, lactose, maltose, maltodextrins, corn syrup solids, honey solids and commercial tablet nutritional supplements; sugar alcohols such as mannitol, sorbitor and xylitol; and, various relatively insoluble excipients, including dicalcium phosphate, calcium sulfate, calcium carbonate, microcrystalline cellulose and other pharmaceutical tableting ingredients.

Lozenges, tablets and troches used in the present invention may differ in shape, size and manufacturing technique. Where the composition is included in tablets for oral use, the pharmaceutically acceptable carrier may further include lactose and corn starch. Lubricating agents (e.g., magnesium stearate, sodium lauryl sulfate and talc) may also be added to the tablets. Tablets may also include excipients (e.g., sodium citrate, calcium carbonate and calcium phosphate), disintegrants (e.g., starch, alginic acid and complex silicates) and binding agents (e.g., polyvinylpyrrolidone, gelatin, PEG-8000 and gum acacia).

For lozenges, a common pharmaceutically acceptable carrier may further include a binder such as PEG-8000. Preferably, the total weight of a lozenge ranges between 1.25 to 10 grams, more preferably, 0.75 to 0.5 grams, to provide a suitable dissolution rate when taken orally.

A composition of the present invention is typically added to PEG-8000 processed fructose to make compressible lozenges; or, the composition is added to crystalline fructose and commercially available, sweet, direct compression products (e.g., Mendell's SUGARTAB™). The mixture should be kept dry, and tableted soon after mixing. The ingredients are mixed and directly pressed into lozenges using conventional pharmaceutical mixing and tableting equipment. The compressive force is preferably sufficient to produce maximum hardness throughout the lozenges, to preserve a suitable dissolution rate, and to maximize the efficacy of the lozenges. Dissolution of the lozenges should occur over a sustained period of time, such as 5 to 60 minutes and preferably 10 to 30 minutes.

Tablets and troches can be manufactured using procedures similar to those described above with slight modifications in optional ingredients. Such changes are well within the abilities of one with ordinary skill in the art.

The composition of the present invention may alternatively be formulated in liquid form (e.g., as a syrup, mouth rinse/wash or spray) with a solvent or dispersant such as water, or other suitable liquids and optionally in a pharmaceutically acceptable carrier. Treatment time is preferably 10 to 60 seconds and more preferably 20 to 30 seconds, so as to permit a prolonged contact of the composition with mouth and throat tissues.

The composition of the present invention may also be formulated in a chewable form, such as soft candy, gum drops, liquid-filled candies, chewing gum bases, toothpastes and gargling solutions.

The composition may further be formulated in capsule form, with or without diluents. Useful diluents for capsules include Mannitol, lactose and dried corn starch. When suspensions are employed, emulsifying and/or suspending agents may be used. Furthermore, solid compositions, including one or more of the lozenge ingredients described above, may be used in soft and hard gelatin capsules.

The composition may still be further formulated into a nasal aerosol or inhalant composition. Such a composition may be prepared using well-known techniques. For nasal/ inhalant compositions, suitable carriers include, without limitation, the following: saline with one or more preservatives; absorption promoters to enhance bioavailability; fluorocarbons; and/or conventional solubilizing or dispersion agents.

The following are non-limiting examples of formulations of the present invention.

Formulation 1, which is a rinse and comprises the following: 250 mL to 1500 mL of water, typically E.C.A. processed water, as an aqueous solution containing cesium salt (50 to 150 mg); potassium salt (50 to 300 mg); and, magnesium salt (25 to 150 mg).

Formulation 2, which is a rinse and comprises the following: 250 mL to 1500 mL of water, typically E.C.A. processed water, as an aqueous solution containing cesium chloride (50 to 150 mg); potassium citrate (50 to 300 mg); and magnesium citrate or ascorbate (15 to 150 mg).

Formulation 3, which is a rinse and comprises the following: 250 mL to 1500 mL of water, typically E.C.A. processed water, as an aqueous solution containing cesium chloride (100 mg); potassium citrate (100 to 150 mg); and magnesium, citrate or ascorbate (50 to 75 mg).

Formulation 4, which is a capsule or tablet or capsule comprising the following: cesium or rubidium salts ranging in amount from 10 mg to 250 mg. Other ingredients that may be included are, without limitation, cesium citrate, rubidium citrate, potassium (preferably as phosphate, citrate gluconate and acetate), calcium (carbonate), magnesium citrate, manganese, iodine, selenium (Selenomethionine), vanadium (vanadyl sulfate), zinc gluconate, Vitamin D3 (Cholecalciferol)

Vitamin A, Vitamin C (buffered L-ascorbic acid), malic acid, Co-enzyme Q 10 (ubiquinone), B3 (methyl nicotinate), B6 (pyridoxine), B12 (cyanocobalamin), and superoxide dismutase.

Formulation 5, which is a capsule or tablet comprising the following: cesium and/or rubidium salts ranging in amount from 20 mg to over 100 mg, generally 25 mg to 100 mg. Other ingredients that may be included are (maximal dosages for a 70 kg male per 24 hours): iodine 150 to 400 mcg.; selenium (Selenomethionine) 50 to 200 mcg; vanadium (vanadyl sulfate) 2 to 10 mg; zinc gluconate 50 to 200 mg; Vitamin D3 2,000 to 4,000 IU (cholecalciferol); Vitamin A 2,000 to 5,000 IU; Vitamin C (buffered L-ascorbic acid); 500 to 2,000 mg; malic acid 3 to 5 mg; Co-enzyme Q 10 (ubiquinone) 25 to 50 mg; B3 (methyl nicotinate) 5 to 20 mg; B6 (pyridoxine) 25-100 mg; B12 (cyanocobalamin) 20 to 50 mcg; superoxide dismutase 100 to 2,000 mg. Preferably 250 to 500 mg.

Formulation 6, which is a capsule or tablet comprising the following: cesium salt (50 to 150 mg); potassium salt (50 to 300 mg); and, magnesium salt (25 to 150 mg).

Formulation 7, which is a capsule or tablet comprising the following: cesium chloride (50 to 150 mg); potassium citrate (50 to 300 mg); and magnesium citrate or ascorbate (15 to 150 mg).

Formulation 8, which is a capsule or tablet comprising the following: cesium chloride (100 mg); potassium citrate (100 to 150 mg); and magnesium citrate or ascorbate (50 to 75 mg).

Formulation 9, which is an effervescent tablet or granules comprising the following: at least one acid component and one gas-evolving component of alkali hydrogen carbonate, alkali carbonate, and/or alkaline-earth carbonate particles evolving gas under the action of acid, as well as of active salts of cesium and rubidium, with potassium, magnesium and other electrolytes, fragrances, colorants, plant extracts, vitamins, minerals and trace minerals admixed as needed, with at least one acid component combined with at least one of the following compounds: alkali carbonate, alkali hydrogen carbonate, alkaline-earth carbonate, alkaline-earth oxide, hydrocolloid, or mixtures of glycols, etc. Additional calcium carbonate can be added to this phase in order to obtain a higher calcium dose if required.

Formulation 10, which is an effervescent tablet or granules comprising the following: cesium salt (10 to 50 mg); potassium salt (10 to 100 mg), and magnesium salt (25 to 150 mg).

Formulation 11, which is an effervescent tablet or granules comprising the following: cesium chloride (10 to 50 mg); potassium citrate (10 to 100 mg); and magnesium citrate or ascorbate (5 to 50 mg).

Formulation 12, which is an effervescent tablet or granules comprising the following: cesium chloride (100 mg); potassium citrate (20 to 25 mg); and magnesium citrate or ascorbate (10 to 15 mg).

Formulation 13, which is a lozenge comprising the following: cesium chloride (50 to 150 mg); potassium citrate (10 to 60 mg); and magnesium citrate or ascorbate (3 to 30 mg).

Formulation 14, which is a lozenge comprising the following: cesium salt (10 to 30 mg); potassium salt (10 to 60 mg); and, magnesium salt (5 to 30 mg).

The present invention also provides systems for preventing and reducing the duration of common colds and flu and for reducing the severity of common cold and flu symptoms. Non-limiting examples of such systems include those discussed below.

In a system aspect of the present invention, a container (e.g., bottle, can, etc) containing a rinse is provided. The container includes instructions, either attached to the container or provided with it, regarding the proper way to ingest the rinse for optimal effect regarding preventing, alleviating or eliminating cold and/or flu symptoms. Preferably, the container includes instructions related to the appropriate amount of rinse to ingest to achieve optimal effect (e.g., from 5 mg to 50 mg of cesium and/or rubidium salt per 24 hour period). The container optionally includes instructions on the combination of diet modification and the (rinse) to optimally modulate/elevate pH.

In another system aspect of the present invention, capsules or tablets are included in a container (e.g., bottle). The container includes instructions, either attached to the container or included with it, regarding the proper way to ingest the capsules or tablets for optimal effect regarding preventing, alleviating or eliminating cold and/or flu symptoms. Preferably, the container includes instructions related to the appropriate dosage to ingest to achieve optimal effect. The container optionally includes instructions on the combination of diet modification and the tablets/capsules to optimally elevate/modulate the pH/reducing acidity.

In another system aspect of the present invention, lozenges are included in a container (e.g., paper or plastic wrap). The container includes instructions, either attached to the container or included with it, regarding the proper way to ingest the capsules or tablets for optimal effect regarding preventing, alleviating or eliminating cold and/or flu symptoms. Preferably, the container includes instructions related to the appropriate dosage to ingest to achieve optimal effect (e.g., cesium or rubidium salts ingestion ranging from 10 mg to 100 mg per 24 hours, up to a total dose of about 250 mg per 24 hours). The container optionally includes instructions on the combination of diet modification and the lozenges to optimally elevate the oral pH/reducing the acidity.

The method of the present invention involves the administration of a composition of the present invention to a patient that anticipates or suffers from one or more of the following: a common cold, flu, a sore throat, muscle soreness, fever, congestion, laryngitis, mucositis and mucous membrane inflammation, i.e. pain. The composition may be administered 1-10 times per 24 hours, as needed, more preferably 2-4 times per 24 hours.

The method of the present invention treats cold/flu symptoms, but it may be continued to provide substantial, ongoing relief. The method may further be used to prevent (i.e., prophylaxis) the symptoms of a common cold, flu, muscle soreness, fever, sore throat, pain congestion, laryngitis, and mucous membrane inflammation by prophylactic administration of an effective amount of the composition.

Cesium and rubidium ions used in the present are separate and distinct from man-made isotopes of cesium and rubidium.

### EXAMPLES

### Example 1

### A Composition of the Present Invention

A composition of the present invention formulated in the form of lozenges is prepared using the procedure described above. The active ingredients of the lozenge are listed below:

### 50 ppm to 500,000 ppm cesium ion source

### Example 2

### Treatment of Sore Throat

Subjects suffering from sore throats are provided with a lozenge according to Example 1. The subjects are instructed to take 1 lozenge every two hours and to hold the lozenge in his or her mouth for 15-30 minutes, until the lozenge dissolves.

### Example 3

### In Vitro Testing of Virucidal Activity

The protocol for testing virucidal activity uses human rhinovirus 16 (hereinafter “HRV-16”) as the target virus and the MRC-5 cell line as the host cell for the virus. See Jacobs et al., Characteristics of Human Diploid MRC-5, Nature (London), 227:168-170 (1970). Residual virus infectivity is titrated following incubation of the test substances with the virus. Cytopathic effect (CPE) induced by virus replication is visually scored through microscopic observation—i.e., by observing ballooning/rounded cells in the MRC-5 culture.

A composition of the present invention is employed at an initial dilution of 1/20 and then further diluted by serial dilutions in saline. The diluted compositions are incubated with HRV-16 for a set time period; the reaction is then terminated by adjustment to a neutral pH with cell infection media. The resultant solution is titrated out on MRC-5 cells at a dilution of 1/10 across a testing plate to carry out cell infection. Each plate houses a virus control, which contains only HRV-16 infected MRC-5 cells and a cell control that only contains uninfected MRC-cells.

The plates are further incubated for 4 days after infection. Residual and viral infectivity is measured using the assay discussed above.

### Example 4

### In Vitro Testing of Virustatic Activity

The protocol for testing virucidal activity uses human rhinovirus 16 (hereinafter “HRV-16”) as the target virus and a rhinovirus sensitive Hela cell line related to human tissues as the host cell. See Conant et al., Basis for a Numbering System. I. Hela Cells for Propagation and Serologic Procedure, J. Immunol., 100:107-113 (1968).

A composition of the present invention is dissolved in infection media to the following dilutions: 1/20, 1/40, 1/80, 1/160 and 1/320. The dilutions are incubated on plates of MRC-5 cells for 30 min. at 37° C. (5% CO₂). After the incubation period, each composition dilution with MRC-5 cells in a well of plates is subjected to HRV-16 at a known titer of 2.30 (−log 10 TCID50). Each plate houses a virus control (the Hela cells infected with HRV-16 viruses and without the composition of the present invention),a cell control (Hela cells only) and the test composition controls at the various dilutions (Hela cells with the test substance only). All other samples on the plate contain Hela cells infected with HRV-16 viruses and the composition at different dilutions. The plates are further incubated for 4 days after infection.

Residual virus infectivity following incubation with the composition and virus is titrated on the Hela cell line for rhinovirus growth. This is done by measuring the cytopathic effect (CPE) induced by the virus using the following procedure: The remaining viable Hela cells, after incubation with the composition, are stained with crystal violet solution. Excess crystal violet is removed by washing, and the crystal violet stained cells are solubilized using a mixture of methanol and acetic acid. The absorbance of the solution is measured at 540 nm in an ELISA plate reader. The level of virus induced CPE is inversely proportional to the absorbance.

The results generated from the crystal violet assay enable the toxic concentration and effective concentration of the composition to be determined by fitting an equation—y=mx+c. In the equation, x is the dilution of the composition; y is the percentage of toxicity of the composition to the cells.

1. A composition for treating a common cold or flu in a patient, wherein the composition comprises: 50 ppm to 500,000 ppm of a cesium ion source, a rubidium ion source or a mixture of the two.

2. The composition according to claim 1, wherein the composition comprises 50 ppm to 500,000 ppm of a cesium ion source.

3. The composition according to claim 1, wherein the composition comprises 50 ppm to 500,000 ppm of a rubidium ion source.

4. The composition according to claim 2, wherein the composition further comprises a structuring agent selected from gum tragacanth, xanthan gum, gum karaya and gum arabic, seaweed derivatives, smectite clays such as diatomaceous earths, bentonite or hectorite, calcium apatite, carboxyvinyl polymers and water-soluble cellulose derivatives such as hydroxyethyl cellulose and sodium carboxymethyl cellulose present in an amount of from 0 to 5 percent by weight of the composition.

5. The composition according to claim 3, wherein the composition further comprises a structuring agent selected from gum tragacanth, xanthan gum, gum karaya and gum arabic, seaweed derivatives, smectite clays such as diatomaceous earths, bentonite or hectorite, calcium apatite, carboxyvinyl polymers and water-soluble cellulose derivatives such as hydroxyethyl cellulose and sodium carboxymethyl cellulose present in an amount of from 0 to 5 percent by weight of the composition.

6. The composition according to claim 2, wherein the composition further comprises a flavoring agent selected from wintergreen oil, oregano oil, bay leaf oil, peppermint oil, spearmint oil, clove oil, sage oil, sassafras oil, cherry oil, lemon oil, orange oil, anise oil, benzaldehyde, bitter almond oil, camphor, cedar leaf oil, marjoram oil, citronella oil, lavender oil, mustard oil, pine oil, pine needle oil, rosemary oil, thyme oil, cinnamon leaf oil, and mixtures thereof present in an amount from 0.05 to 5 percent by weight of the composition.

7. The composition according to claim 3, wherein the composition further comprises a flavoring agent selected from wintergreen oil, oregano oil, bay leaf oil, peppermint oil, spearmint oil, clove oil, sage oil, sassafras oil, cherry oil, lemon oil, orange oil, anise oil, benzaldehyde, bitter almond oil, camphor, cedar leaf oil, marjoram oil, citronella oil, lavender oil, mustard oil, pine oil, pine needle oil, rosemary oil, thyme oil, cinnamon leaf oil, and mixtures thereof present in an amount from 0.05 to 5 percent by weight of the composition.

8. The composition according to claim 2, wherein the composition further comprises an herbal ingredient selected from Allium cepa (red onion), Salvia officinalis (Sage), Sambucus nigra (elderberry), Capsicum (cayenne fruit), Mentha pipenta (peppermint), Thymus serpyllum (thyme), Aconitum napellus (monks hood), Allium sativum (garlic), Anas barbariae (Oscillococcinum), Euphrasia officinalis (eyebright), Gelsemium sempervirens (yellow jasmine), Phytolacca decandra (poke), and Pulsatilla nigricans (wind flower) present at a concentration of 1× to 60 C.

9. The composition according to claim 3, wherein the composition further comprises an herbal ingredient selected from Allium cepa (red onion), Salvia officinalis (Sage), Sambucus nigra (elderberry), Capsicum (cayenne fruit), Mentha pipenta (peppermint), Thymus serpyllum (thyme), Aconitum napellus (monks hood), Allium sativum (garlic), Anas barbariae (Oscillococcinum), Euphrasia officinalis (eyebright), Gelsemium sempervirens (yellow jasmine), Phytolacca decandra (poke), and Pulsatilla nigricans (wind flower) present at a concentration of 1× to 60 C.

10. The composition according to claim 2, wherein the composition further comprises a non-homeopathic drug selected from antihistamines, decongestants, pain relievers, cough suppressants, expectorants, and analgesics.

11. The composition according to claim 3, wherein the composition further comprises a non-homeopathic drug selected from antihistamines, decongestants, pain relievers, cough suppressants, expectorants, and analgesics.

12. The composition according to claim 2, wherein the composition further comprises a zinc salt selected from zincum gluconicum, zinc gluconate, and zinc sulfate present in a concentration ranging from 0.1 percent to 3.0 percent weight/volume of the composition.

13. The composition according to claim 3, wherein the composition further comprises a zinc salt selected from zincum gluconicum, zinc aspartate, zinc citrate, zinc gluconate, and zinc sulfate present in a concentration ranging from 0.1 percent to 3.0 percent weight/volume of the composition.

14. A formulation for treating a common cold in a patient, wherein a composition according to claim 1-13 is formulated as a capsule, tablet or troche.

15. A formulation for treating a common cold in a patient, wherein a composition according to claims 1-13 is formulated as a lozenge or candy.

16. A formulation for treating a common cold in a patient, wherein a composition according to claims 1-13 is formulated as a spray or mouth rinse.

17. A method of treating a patient having a common cold, wherein the method comprises administering a composition according to claims 1-13 to the patient.

18. A method of preventing a subject from contracting a common cold, wherein the method comprises administering a composition according to claims 1-13 to the patient.

19. A kit for treating a common cold or flu in a patient, wherein the kit comprises:
a) 50 ppm to 500,000 ppm of a cesium ion source, a rubidium ion source or a mixture of the two in the form of a capsule, lozenge, trouche, or mouth rinse; and,
b) instructions on how to use the capsule, lozenge, trouch, or mouth rinse to treat the cold or flu.
